(19) United States
(12) Patent Application Publication
(10) Pub. No.: US 2025/0276990 A1
(43) Pub. Date: Sep. 4, 2025

(54) MARINE PLANT SYSTEM AND METHOD FOR CONTROLLING THE SAME

(71) Applicant: TOYOTA JIDOSHA KABUSHIKI KAISHA, Toyota-shi (JP)

(72) Inventor: Tadayoshi SUMIYA, Toyota-shi (JP)

(73) Assignee: TOYOTA JIDOSHA KABUSHIKI KAISHA, Toyota-shi (JP)

(21) Appl. No.: 19/042,317

(22) Filed: Jan. 31, 2025

(30) Foreign Application Priority Data
Mar. 4, 2024 (JP) 2024-031949

(51) Int. Cl. C07H 3/06; C07H 1/08; C25B 1/16; C25B 1/26

(52) U.S. Cl. CPC C07H 3/06; C07H 1/08; C25B 1/16; C25B 1/26

(57) ABSTRACT

A marine plant system according to one aspect of the present disclosure includes: a biochar production apparatus configured to produce biochar from seaweed collected from the sea; an alginate production apparatus configured to produce alginate from the seaweed; a power generation apparatus configured to supply electric power generated using renewable energy to the biochar production apparatus and the alginate production apparatus; and a controller configured to control production of the biochar and production of the alginate. When an amount of the electric power is smaller than a predetermined reference value, the controller selects production of the biochar.

### CROSS REFERENCE TO RELATED APPLICATIONS

This application is based upon and claims the benefit of priority from Japanese patent application No. 2024-031949, filed on Mar. 4, 2024, the disclosure of which is incorporated herein in its entirety by reference.

### BACKGROUND

The present disclosure relates to a marine plant system and a method for controlling the same.

As disclosed in Patent Literature 1, the inventors have developed a marine plant system in which seaweed is grown, carbon dioxide in the air is captured, and various kinds of products are produced from the seaweed.
Patent Literature 1: Japanese Unexamined Patent Application No. 2024-30988

### SUMMARY

The present inventors have studied a marine plant system for producing alginate from seaweed by using electric power generated using renewable energy. By using renewable energy, Life Cycle Assessment (LCA) of alginate to be produced can be reduced.

However, in a case in which, for example, alginate cannot be produced due to a factor such as a lack of electric power, there is a problem that productivity is significantly reduced if the marine plant system is stopped.

The present disclosure has been made in view of the above background, and an object of the present disclosure is to provide a marine plant system that can operate properly and have excellent productivity even in a case where alginate cannot be produced.

A marine plant system according to one aspect of the present disclosure includes: a biochar production apparatus configured to produce biochar from seaweed collected from the sea; an alginate production apparatus configured to produce alginate from the seaweed; a power generation apparatus configured to supply electric power generated using renewable energy to the biochar production apparatus and the alginate production apparatus; and a controller configured to control production of the biochar and production of the alginate, in which, when an amount of the electric power is smaller than a predetermined reference value, the controller selects production of the biochar.

In the marine plant system according to one aspect of the present disclosure, when an amount of electric power is smaller than a predetermined reference value, production of biochar is selected. That is, when alginate cannot be produced due to a lack of electric power, the marine plant system operates properly by selecting production of biochar, whereby it has excellent productivity.

The marine plant system may further include an intermediate material production apparatus configured to produce an intermediate material for producing the alginate, in which, when an amount of the intermediate material is smaller than a predetermined reference value, the controller may select production of the intermediate material. That is, when alginate cannot be produced due to a lack of the intermediate material, the marine plant system operates properly by selecting production of the intermediate material, whereby it has excellent productivity.

The marine plant system may further include: a seawater treatment apparatus configured to separate seawater into fresh water and concentrated seawater; and an electrolysis apparatus configured to electrolyze the concentrated seawater separated by the seawater treatment apparatus, in which the intermediate material production apparatus may produce calcium chloride and sodium hydrogen carbonate from calcium carbonate using the fresh water separated by the seawater treatment apparatus, and hydrogen gas, chlorine gas, and sodium hydroxide generated by the electrolysis apparatus, and the alginate production apparatus may extract, using calcium chloride and sodium hydrogen carbonate produced by the intermediate material production apparatus, alginate from the seaweed and produce calcium alginate. That is, this marine plant system electrolyzes the concentrated seawater separated by the seawater treatment apparatus by an electrolysis apparatus to produce hydrogen gas, chlorine gas, and sodium hydroxide. Therefore, there is no need to purchase hydrochloric acid and sodium hydroxide that are required to produce calcium alginate and to transport them to the marine plant system by a container ship or the like, whereby it is possible to significantly reduce LCA of calcium alginate to be produced.

The marine plant system may further include a storage apparatus configured to store residue generated in the alginate production apparatus; and a boiler configured to burn the residue, in which, when a free space in the storage apparatus is smaller than a predetermined reference value, the controller may select burning of the residue stored in the storage apparatus by a boiler. That is, when alginate cannot be produced due to a lack of a free space in a storage apparatus, the marine plant system may operate properly by selecting burning of the residue stored in the storage apparatus by the boiler, whereby it has excellent productivity.

Calcium carbonate contained in ash generated in the boiler may be supplied to the intermediate material production apparatus. There is no need to purchase calcium carbonate required to produce calcium alginate and transport it to a marine plant system by a container ship or the like, whereby it is possible to reduce LCA of calcium alginate to be produced.

A method for controlling a marine plant system according to one aspect of the present disclosure is a method for controlling the marine plant system, the marine plant system including: a biochar production apparatus configured to produce biochar from seaweed collected from the sea; an alginate production apparatus configured to produce alginate from the seaweed; and a power generation apparatus configured to supply electric power generated using renewable energy to the biochar production apparatus and the alginate production apparatus, in which, when an amount of the electric power is smaller than a predetermined reference value, a controller that controls production of the biochar and production of the alginate selects production of the biochar.

In the method for controlling the marine plant system according to one aspect of the present disclosure, when an amount of the electric power is smaller than a predetermined reference value, production of biochar is selected. That is, when alginate cannot be produced due to a lack of electric power, the marine plant system operates properly by selecting production of biochar, whereby it has excellent productivity.

The marine plant system may further include an intermediate material production apparatus configured to produce an intermediate material for producing the alginate, and, when an amount of the intermediate material is smaller than a predetermined reference value, the controller may select production of the intermediate material. That is, when alginate cannot be produced due to a lack of the intermediate material, the marine plant system operates properly by selecting production of the intermediate material, whereby it has excellent productivity.

The marine plant system may further include: a seawater treatment apparatus configured to separate seawater into fresh water and concentrated seawater; and an electrolysis apparatus configured to electrolyze the concentrated seawater separated by the seawater treatment apparatus, in which the intermediate material production apparatus may produce calcium chloride and sodium hydrogen carbonate from calcium carbonate using the fresh water separated by the seawater treatment apparatus, and hydrogen gas, chlorine gas, and sodium hydroxide generated by the electrolysis apparatus, and the alginate production apparatus may extract, using calcium chloride and sodium hydrogen carbonate produced by the intermediate material production apparatus, alginate from the seaweed and produce calcium alginate. That is, the marine plant system electrolyzes concentrated seawater separated by the seawater treatment apparatus by an electrolysis apparatus, and produces hydrogen gas, chlorine gas, and sodium hydroxide. Therefore, there is no need to purchase hydrochloric acid and sodium hydroxide that are required to produce calcium alginate and to transport them to the marine plant system by a container ship or the like, whereby it is possible to significantly reduce LCA of calcium alginate to be produced.

The marine plant system may further include: a storage apparatus configured to store residue generated in the alginate production apparatus; and a boiler configured to burn the residue, in which, when a free space in the storage apparatus is smaller than a predetermined reference value, the controller may select burning of the residue stored in the storage apparatus by the boiler. That is, when it is determined that alginate cannot be produced due to a lack of the free space in the storage apparatus, the marine plant system operates properly by selecting burning of the residue stored in the storage apparatus by a boiler, whereby it has excellent productivity.

The calcium carbonate contained in ash generated in the boiler may be supplied to the intermediate material production apparatus. There is no need to purchase calcium carbonate required to produce calcium alginate and transport it to the marine plant system by a container ship or the like, whereby it is possible to reduce LCA of calcium alginate to be produced.

According to the present disclosure, it is possible to provide a marine plant system that can operate properly and have excellent productivity even in the case where alginate cannot be produced.

The above and other objects, features and advantages of the present disclosure will become more fully understood from the detailed description given hereinbelow and the accompanying drawings.

### BRIEF DESCRIPTION OF DRAWINGS

FIG. 1 is a block diagram showing a configuration of a marine plant system according to a first embodiment;

FIG. 2 is a block diagram showing one example of a configuration of a seawater treatment apparatus 10;

FIG. 3 is a schematic diagram showing one example of a configuration of an electrolysis apparatus 20;

FIG. 4 is a flowchart showing a method for controlling the marine plant system according to the first embodiment;

FIG. 5 is a flowchart showing a method for producing calcium alginate according to the first embodiment; and

FIG. 6 is a flowchart showing one example of details of Step ST4 in FIG. 5.

### DESCRIPTION OF EMBODIMENTS

Hereinafter, with reference to the drawings, specific embodiments of the present disclosure will be described in detail. However, the present disclosure is not limited to the following embodiments. Further, for the sake of clarification of the description, the following descriptions and the drawings are simplified as appropriate.

### First Embodiment

### <Configuration of Marine Plant System>

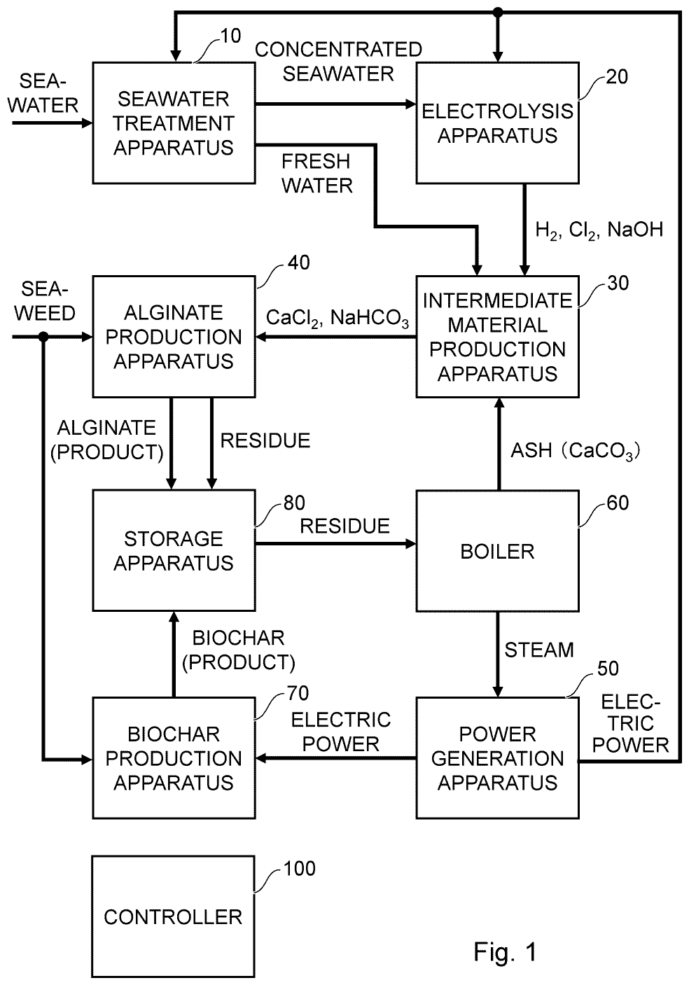

First, by referring to FIG. 1, a configuration of a marine plant system according to a first embodiment will be described. FIG. 1 is a block diagram showing a configuration of the marine plant system according to the first embodiment.

As shown in FIG. 1, the marine plant system according to this embodiment includes a seawater treatment apparatus 10, an electrolysis apparatus 20, an intermediate material production apparatus 30, an alginate production apparatus 40, a power generation apparatus 50, a boiler 60, a biochar production apparatus 70, a storage apparatus 80, and a controller 100.

As shown in FIG. 1, the seawater treatment apparatus 10, which is, for example, a seawater desalination apparatus, separates seawater into fresh water and concentrated seawater. That is, the seawater treatment apparatus 10 produces fresh water and concentrated seawater from seawater. The fresh water separated by the seawater treatment apparatus 10 is supplied to the intermediate material production apparatus 30. On the other hand, the concentrated seawater separated by the seawater treatment apparatus 10 is supplied to the electrolysis apparatus 20. While concentrated seawater separated by the seawater desalination apparatus has generally been, for example, discharged into the sea, in the marine plant system according to this embodiment, this concentrated seawater is efficiently used.

Note that the fresh water separated by the seawater treatment apparatus 10 can be used for various applications. Therefore, this fresh water may be supplied not only to the intermediate material production apparatus 30 but also to the electrolysis apparatus 20, the alginate production apparatus 40, and the like.

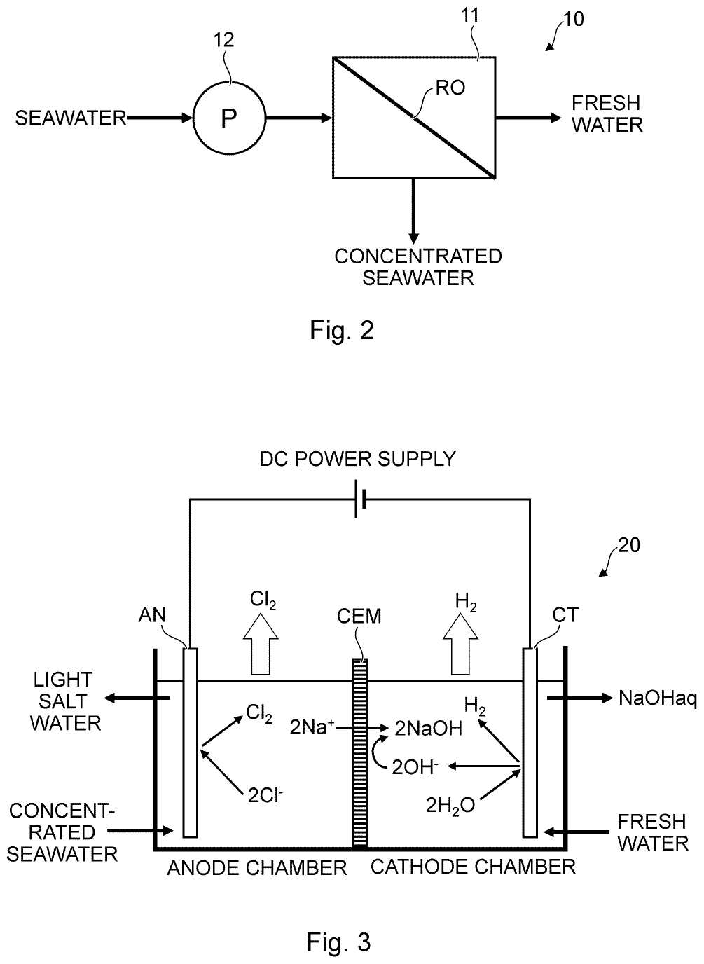

FIG. 2 is a block diagram showing one example of a configuration of the seawater treatment apparatus 10. The seawater treatment apparatus 10 shown in FIG. 2 includes a reverse osmosis membrane module 11 and a pump 12. As shown in FIG. 2, the reverse osmosis membrane module 11 includes a reverse osmosis membrane RO. The seawater is pumped to the reverse osmosis membrane module 11 from the pump 12, and the seawater is separated into fresh water that has passed through the reverse osmosis membrane RO and concentrated seawater that does not pass through the reverse osmosis membrane RO.

In the seawater treatment apparatus 10, by using the reverse osmosis membrane RO, seawater can be separated into fresh water and concentrated seawater easily and inexpensively.

Note that a method for separating seawater into fresh water and concentrated seawater is not particularly limited, and an ion exchange membrane may be, for example, used in place of the reverse osmosis membrane RO. Further, FIG. 2 schematically shows separation of seawater into fresh water and concentrated seawater by the reverse osmosis membrane RO, and does not necessarily shows the actual configuration of the seawater treatment apparatus 10.

As shown in FIG. 1, the electrolysis apparatus 20 electrolyzes the concentrated seawater separated by the seawater treatment apparatus 10. By electrolyzing the concentrated seawater in the electrolysis apparatus 20, hydrogen gas (H₂), chlorine gas (Cl₂), and sodium hydroxide (NaOH) are generated. H₂, Cl₂, and NaOH generated by the electrolysis apparatus 20 are supplied to the intermediate material production apparatus 30.

FIG. 3 is a schematic diagram showing one example of a configuration of the electrolysis apparatus 20. The electrolysis apparatus 20 shown in FIG. 3 includes a DC power supply, an anode AN, a cathode CT, and a cation exchange membrane CEM. As shown in FIG. 3, an anode chamber and a cathode chamber are divided from each other by the cation exchange membrane CEM.

As shown in FIG. 3, concentrated seawater (NaCl) is supplied to the anode chamber. In the anode chamber, chloride ions (Cl⁻) release electrons to the anode AN, which causes chlorine gas (Cl₂) to be generated. Sodium ions (Na⁺) pass through the cation exchange membrane CEM to move to the cathode chamber. As a result, the concentration of NaCl is reduced in the anode chamber and light salt water is discharged from the anode chamber.

On the other hand, fresh water is supplied to the cathode chamber. This fresh water is, for example, the fresh water separated by the seawater treatment apparatus 10. In the cathode chamber, water (H₂O) receives electrons from the cathode CT, which causes hydrogen gas (H₂) and hydroxide ions (OH⁻) to be generated. Then hydroxide ions (OH⁻) are coupled to sodium ions (Na⁺), which causes sodium hydroxide (NaOH) to be generated.

That is, in the entire electrolysis apparatus 20, the reaction shown by the following chemical reaction formula (1) occurs.

2NaCl+2H₂O→Cl₂+H₂+2NaOH (1)

A dilute aqueous sodium hydroxide solution may be supplied to the cathode chamber instead of supplying fresh water thereto.

The fresh water separated by the seawater treatment apparatus 10, and H₂, Cl₂, and NaOH generated by the electrolysis apparatus 20 are supplied to the intermediate material production apparatus 30. Then, the intermediate material production apparatus 30 produces calcium chloride (CaCl₂) and sodium hydrogen carbonate (NaHCO₃) from calcium carbonate (CaCO₃) using the above fresh water and H₂, Cl₂, and NaOH.

First, as shown in the following chemical reaction formula (2), hydrogen chloride (HCl) is produced from H₂ and Cl₂.

H₂+Cl₂→2HCl (2)

Then, after HCl is absorbed by fresh water, hydrochloric acid (HClaq) is obtained.

Next, as shown in the following chemical reaction formula (3), hydrochloric acid and CaCO₃ are reacted to obtain CaCl₂.

2HClaq+CaCO₃→H₂O+CO₂+CaCl₂ (3)

Next, as shown in the following chemical reaction formula (4), carbon dioxide (CO₂) generated in the chemical reaction formula (3) is reacted with an aqueous sodium hydroxide solution (NaOHaq) to obtain an aqueous sodium hydrogen carbonate solution (NaHCO₃aq).

NaOHaq+CO₂→NaHCO₃aq (4)

The alginate production apparatus 40 extracts, by using CaCl₂ and NaHCO₃ produced by the intermediate material production apparatus 30, alginate from seaweed collected from the sea and produces calcium alginate.

First, as shown in the following chemical reaction formula (5), NaHCO₃ produced by the intermediate material production apparatus 30 is added to the seaweed, and alginate in the seaweed is extracted as soluble sodium alginate. Alginic acid in the seaweed is coupled, for example, to multivalent cations such as Ca²⁺ to form insoluble alginate.

Calcium alginate (in the seaweed)+2NaHCO₃→2 sodium alginate+Ca(HCO₃)₂ (5)

Next, as shown in the following chemical reaction formula (6), CaCl₂ produced by the intermediate material production apparatus 30 is added to the sodium alginate extraction liquid to precipitate insoluble calcium alginate.

2 sodium alginate+CaCl₂→calcium alginate+2NaCl (6)

As a result of the above-described procedure, calcium alginate, which is a product, can be obtained.

Note that the details of various kinds of processing in the alginate production apparatus 40 will be described later.

Further, dilute sulfuric acid may be added to the sodium alginate extraction liquid instead of adding CaCl₂) thereto, and free alginate may be precipitated in place of calcium alginate.

As shown in FIG. 1, the power generation apparatus 50 supplies electric power generated using renewable energy. The power generation apparatus 50 is any one of a solar power generation apparatus, a wind power generation apparatus, a hydrogen power generation apparatus and the like or a combination thereof. It is difficult to stably supply electric power in the solar power generation apparatus and the wind power generation apparatus as they are affected by weather, and thus use of them may result in a lack of electric power. Further, the hydrogen power generation apparatus is, for example, a fuel cell, and may generate electric power by using H₂ generated by the electrolysis apparatus 20.

Further, the power generation apparatus 50 shown in FIG. 1 includes a turbine (not shown) that generates power by steam generated by a boiler 60 that will be described later. However, this turbine may not be necessarily provided. Further, the power generation apparatus 50 may include a battery (not shown) that stores the generated electric power.

As shown in FIG. 1, the electric power generated by the power generation apparatus 50 is supplied, for example, to the seawater treatment apparatus 10, the electrolysis apparatus 20, the biochar production apparatus 70, and the like.

More specifically, the electric power generated by the power generation apparatus 50 is supplied, for example, to the pump 12 of the seawater treatment apparatus 10 shown in FIG. 2, the DC power supply of the electrolysis apparatus 20 shown in FIG. 3, and a heater (not shown) of a biochar production apparatus 70 that will be described later. According to these configurations, it is possible to reduce LCA of calcium alginate and biochar to be produced.

As shown in FIG. 1, the boiler 60 is an apparatus that burns the residue generated in the alginate production apparatus 40 (seaweed after alginate is extracted). Calcium carbonate (CaCO₃) contained in the residue burned in the boiler 60, i.e., ash, is supplied to the intermediate material production apparatus 30.

Therefore, there is no need to purchase CaCO₃ that is necessary to produce calcium alginate and transport it to a marine plant system by a container ship or the like, and it is possible to further reduce LCA of calcium alginate to be produced.

Further, by burning the residue in the boiler 60, it is possible to reduce the amount of the residue stored in the storage apparatus 80, secure the free space, and reduce an amount of waste.

Further, the power generation apparatus 50 can rotate the turbine by steam generated in the boiler 60 to generate electric power. By using this electric power, it is possible to reduce LCA of calcium alginate and biochar to be produced.

The biochar production apparatus 70 produces biochar from the seaweed collected from the sea. More specifically, the biochar production apparatus 70 heats dried seaweed to about, for example, 300° C. by a heater (not shown) in a low-oxygen atmosphere. The low-oxygen atmosphere is an atmosphere in which an oxygen concentration is lower than that in the atmosphere, and is, for example, an atmosphere in which the oxygen concentration is 1 volume % or less.

An amount of electric power to be consumed in the production of biochar is smaller than that in the production of alginate, and an amount of residue to be generated in the production of biochar is significantly smaller than that in the production of alginate.

The storage apparatus 80 stores, for example, calcium alginate and biochar, which are products, the residue that is generated during production, and the like. The storage apparatus 80 may further store NaOH generated by the electrolysis apparatus 20, intermediate materials such as HClaq, CaCl₂, and NaHCO₃ produced by the intermediate material production apparatus 30, the residue burned in the boiler 60, i.e., ash (containing CaCO₃), etc.

The controller 100 controls production of biochar and production of alginate and controls, for example, the entire marine plant system. More specifically, the controller 100 controls allocation of production of biochar by the biochar production apparatus 70 and production of alginate by the alginate production apparatus 40 based on predetermined determination conditions. The predetermined determination conditions include, for example, an amount of electric power that is available, an amount of the intermediate materials, a free space in the storage apparatus 80, and so on. The amount of electric power that is available is, for example, a total of an amount of electric power stored in a battery provided in the power generation apparatus 50 and a predicted amount of power to be generated by the power generation apparatus 50.

Here, the controller 100 determines whether or not alginate can be produced based on the above-described predetermined determination conditions, and selects, when it is determined that alginate can be produced, production of alginate. That is, in the marine plant system according to this embodiment, priority is given to the production of alginate.

On the other hand, the controller 100 selects, when it is determined that alginate cannot be produced due to a lack of electric power, production of biochar where an amount of electric power to be consumed is smaller. Even in the case where alginate cannot be produced due to a lack of electric power, biochar is produced without stopping the marine plant system, whereby it has excellent productivity.

Further, when the controller 100 determines that there is a sufficient amount of electric power but alginate cannot be produced due to a lack of intermediate materials (e.g., HClaq, CaCl₂, and NaHCO₃), the controller 100 selects production of the intermediate materials. By producing the intermediate materials, the problem of the lack of the intermediate materials can be solved, and alginate can be produced. In this manner, even in the case where alginate cannot be produced, the intermediate materials are produced without stopping the marine plant system, whereby excellent productivity can be achieved.

Further, when the controller 100 determines that there is a sufficient amount of electric power but alginate cannot be produced due to a lack of a free space, the controller 100 selects burning of the residue stored in the storage apparatus 80 by the boiler 60. By burning the residue by the boiler 60, the problem of the lack of the free space can be solved, and alginate can be produced. In this manner, even in the case where alginate cannot be produced, the residue is burned by the boiler 60 without stopping the marine plant system, whereby excellent productivity can be achieved.

As described above, even in a case where it is determined that alginate cannot be produced, the marine plant system according to this embodiment properly operates in accordance with its cause, whereby it has excellent productivity. On the other hand, the marine plant system according to this embodiment preferentially produces alginate.

Further, in the marine plant system according to this embodiment, the concentrated seawater separated by the seawater treatment apparatus 10 is electrolyzed by the electrolysis apparatus 20, and HCl and NaOH are produced.

That is, there is no need to purchase HCl and NaOH that are necessary to produce calcium alginate and transport them to the marine plant system by a container ship or the like, whereby it is possible to significantly reduce LCA of calcium alginate to be produced.

Specifically, in the production of calcium alginate according to related art, an amount of CO₂ emissions per kg of product is about 3.0-4.5 kg-CO₂/kg-product. In the marine plant system according to this embodiment, an amount of CO₂ emissions per kg of product is 1.0/kg-product, that is, it can be significantly reduced by about ⅓-¼ or less.

In the marine plant system according to this embodiment, the alginate production apparatus 40, the power generation apparatus 50, the biochar production apparatus 70, and the controller 100 shown in FIG. 1 are absolutely necessary. On the other hand, in the marine plant system according to this embodiment, the seawater treatment apparatus 10, the electrolysis apparatus 20, the intermediate material production apparatus 30, the boiler 60, and the storage apparatus 80 shown in FIG. 1 are not absolutely necessary.

### <Method for Controlling Marine Plant System>

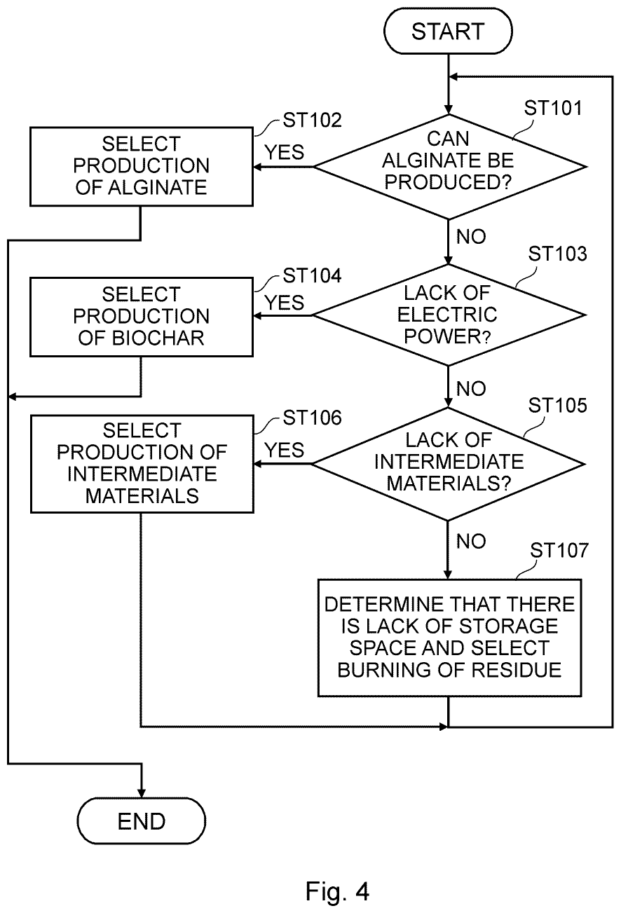

Next, by referring to FIG. 4, a control method by the controller 100 of the marine plant system according to the first embodiment will be described. FIG. 4 is a flowchart showing a method for controlling the marine plant system according to the first embodiment. In the description of FIG. 4, FIG. 1 is referred to as appropriate.

First, as shown in FIG. 4, the controller 100 determines whether or not alginate can be produced based on the aforementioned predetermined determination conditions (Step ST101). In the method for controlling the marine plant system shown in FIG. 4, there are three determination conditions; an amount of electric power that is available, an amount of intermediate materials, and a free space. When the controller 100 determines that alginate can be produced (YES in Step ST101), the controller 100 selects production of alginate (Step ST102). Then the controller 100 ends the control operation.

On the other hand, when the controller 100 determines that alginate cannot be produced (NO in Step ST101), the controller 100 determines whether or not a lack of electric power that is available is the reason why alginate cannot be produced (Step ST103). When it is determined that the amount of electric power that is available is smaller than a predetermined reference value and there is a lack of electric power (YES in Step ST103), the controller 100 selects production of biochar (Step ST104). Then, the controller 100 ends the control operation.

On the other hand, when it is determined that the amount of electric power that is available is equal to or greater than the predetermined reference value and there is a sufficient amount of electric power (NO in Step ST103), the controller 100 determines whether or not a lack of intermediate materials is the reason why alginate cannot be produced (Step ST105). When it is determined that the amount of intermediate materials is smaller than the predetermined reference value and there is a lack of the intermediate materials (YES in Step ST105), the controller 100 selects production of the intermediate materials (Step ST106). After the intermediate materials are produced, the controller 100 updates the amount of the intermediate materials that are available, and the process returns to Step ST101, where it is determined again whether or not alginate can be produced.

On the other hand, when it is determined that the amount of the intermediate materials is equal to or greater than the predetermined reference value and there is a sufficient amount of intermediate materials (NO in Step ST105), the controller 100 determines that the lack of the free space is the reason why alginate cannot be produced (Step ST107). That is, when the free space is smaller than the predetermined reference value and there is a lack of free space, the controller 100 selects burning of the residue stored in the storage apparatus 80 by the boiler 60.

After the residue stored in the storage apparatus 80 is transferred to the boiler 60, the controller 100 updates a free space that is available, and the process returns to Step ST101, where it is determined again whether or not alginate can be produced. Here, when it is determined that alginate can be produced, alginate may be produced while burning the residue by the boiler 60.

As described above, in the method for controlling the marine plant system according to this embodiment, it is first determined whether or not alginate can be produced, and alginate is preferentially produced. On the other hand, even in a case where it is determined that alginate cannot be produced, the marine plant system according to this embodiment properly operates in accordance with its cause, whereby it has excellent productivity.

While there are three conditions for determining whether or not alginate can be produced: an amount of electric power that is available, an amount of intermediate materials, and a free space in the method for controlling the marine plant system shown in FIG. 4, other conditions may be included therein. On the other hand, while the amount of electric power that is available is absolutely necessary as the determination conditions, the amount of the intermediate materials that are available and the free space are not absolutely necessary as the determination conditions. Further, it can be determined whether or not there is a lack of free space that is available before it is determined whether or not there is a lack of the amount of the intermediate materials that are available.

### <Method for Producing Calcium Alginate>

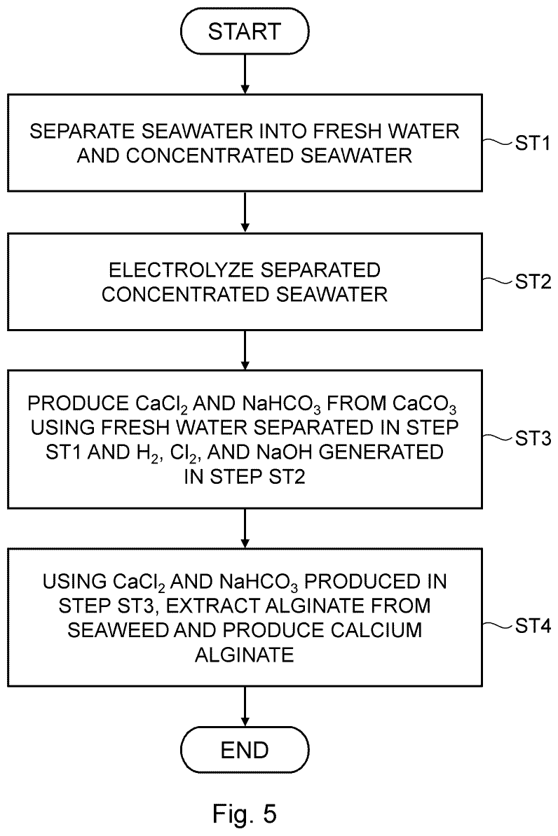

Next, by referring to FIG. 5, a method for producing calcium alginate according to the first embodiment will be described. FIG. 5 is a flowchart showing the method for producing calcium alginate according to the first embodiment. In the description of FIG. 5, FIG. 1 is referred to as appropriate.

First, as shown in FIG. 5, in the seawater treatment apparatus 10 shown in FIG. 1, seawater is separated into fresh water and concentrated seawater (Step ST1).

Next, as shown in FIG. 5, the electrolysis apparatus 20 shown in FIG. 1 electrolyzes the concentrated seawater that has been separated to generate H₂, Cl₂, and NaOH (Step ST2).

Next, as shown in FIG. 5, the intermediate material production apparatus 30 shown in FIG. 1 produces CaCl₂) and NaHCO₃ from CaCO₃ using the fresh water separated in Step ST1 and H₂, Cl₂, and NaOH generated in Step ST2 (Step ST3).

Lastly, as shown in FIG. 5, in the alginate production apparatus 40 shown in FIG. 1, using CaCl₂) and NaHCO₃ produced in Step ST3, alginate is extracted from the seaweed and calcium alginate is produced (Step ST4).

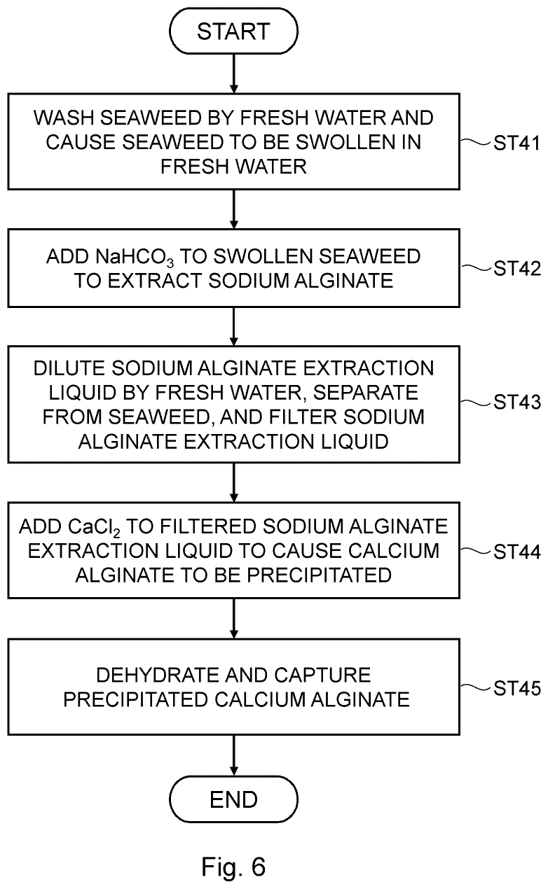

Next, by referring to FIG. 6, details of Step ST4 in FIG. 5 will be described. FIG. 6 is a flowchart showing one example of the details of Step ST4 in FIG. 5.

First, as shown in FIG. 6, the alginate production apparatus 40 washes seaweed collected from the sea by fresh water and causes this seaweed to be swollen in the fresh water (Step ST41). Here, the seaweed may be crushed in advance before Step ST41.

Next, as shown in FIG. 6, in the alginate production apparatus 40, NaHCO₃ is added to the seaweed that is cleaned and swollen to extract sodium alginate (Step ST42). Here, alginic acid contained in the seaweed is coupled to multivalent cations such as Ca²⁺ to form insoluble alginate. Therefore, NaHCO₃ is added to the seaweed and Na is caused to exchange ions with multivalent cations such as Ca²⁺ to extract an aqueous solution containing water-soluble sodium alginate. This aqueous solution is called a sodium alginate extraction liquid.

Next, as shown in FIG. 6, the alginate production apparatus 40 dilutes the sodium alginate extraction liquid by adding fresh water thereto, separates the sodium alginate extraction liquid from the seaweed, and filters the sodium alginate extraction liquid (Step ST43). Here, the fresh air to be added is, for example, the fresh water separated by the seawater treatment apparatus 10. Since viscosity is reduced by dilution, it becomes easy to separate the sodium alginate extraction liquid from the seaweed and filter the sodium alginate extraction liquid.

Next, as shown in FIG. 6, the alginate production apparatus 40 adds CaCl₂ produced by the intermediate material production apparatus 30 to the filtered sodium alginate extraction liquid to cause insoluble calcium alginate to be precipitated (Step ST44).

Lastly, as shown in FIG. 6, the alginate production apparatus 40 captures dehydrated and precipitated calcium alginate (Step ST45).

As a result of the aforementioned series of processing, the alginate production apparatus 40 extracts alginate from seaweed and produces calcium alginate.

The present disclosure promotes use of blue carbon (seaweed) and contributes to carbon neutral, decarbonization, and Sustainable Development Goals (SDGs).

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A marine plant system comprising:
a biochar production apparatus configured to produce biochar from seaweed collected from the sea;
an alginate production apparatus configured to produce alginate from the seaweed;
a power generation apparatus configured to supply electric power generated using renewable energy to the biochar production apparatus and the alginate production apparatus; and
a controller configured to control production of the biochar and production of the alginate,
wherein, when an amount of the electric power is smaller than a predetermined reference value, the controller selects production of the biochar.

2. The marine plant system according to claim 1, further comprising an intermediate material production apparatus configured to produce an intermediate material for producing the alginate,
wherein, when an amount of the intermediate material is smaller than a predetermined reference value, the controller selects production of the intermediate material.

3. The marine plant system according to claim 2, further comprising:
a seawater treatment apparatus configured to separate seawater into fresh water and concentrated seawater; and
an electrolysis apparatus configured to electrolyze the concentrated seawater separated by the seawater treatment apparatus, wherein
the intermediate material production apparatus produces calcium chloride and sodium hydrogen carbonate from calcium carbonate using the fresh water separated by the seawater treatment apparatus, and hydrogen gas, chlorine gas, and sodium hydroxide generated by the electrolysis apparatus, and
the alginate production apparatus extracts, using calcium chloride and sodium hydrogen carbonate produced by the intermediate material production apparatus, alginate from the seaweed and manufactures calcium alginate.

4. The marine plant system according to claim 1, further comprising:
a storage apparatus configured to store residue generated in the alginate production apparatus; and
a boiler configured to burn the residue,
wherein, when a free space in the storage apparatus is smaller than a predetermined reference value, the controller selects burning of the residue stored in the storage apparatus by the boiler.

5. The marine plant system according to claim 4, wherein calcium carbonate contained in ash generated in the boiler is supplied to the intermediate material production apparatus.

6. A method for controlling a marine plant system, the marine plant system comprising:
a biochar production apparatus configured to produce biochar from seaweed collected from the sea;
an alginate production apparatus configured to produce alginate from the seaweed; and
a power generation apparatus configured to supply electric power generated using renewable energy to the biochar production apparatus and the alginate production apparatus,
wherein when an amount of the electric power is smaller than a predetermined reference value, a controller that controls production of the biochar and production of the alginate selects production of the biochar.

7. The method for controlling the marine plant system according to claim 6, wherein
the marine plant system further comprises an intermediate material production apparatus configured to produce an intermediate material for producing the alginate, and
when an amount of the intermediate material is smaller than a predetermined reference value, the controller selects production of the intermediate material.

8. The method for controlling the marine plant system according to claim 7, wherein
the marine plant system further comprises:
a seawater treatment apparatus configured to separate seawater into fresh water and concentrated seawater; and
an electrolysis apparatus configured to electrolyze the concentrated seawater separated by the seawater treatment apparatus,
the intermediate material production apparatus produces calcium chloride and sodium hydrogen carbonate from calcium carbonate using the fresh water separated by the seawater treatment apparatus, and hydrogen gas, chlorine gas, and sodium hydroxide generated by the electrolysis apparatus, and
the alginate production apparatus extracts, using calcium chloride and sodium hydrogen carbonate produced by the intermediate material production apparatus, alginate from the seaweed and manufactures calcium alginate.

9. The method for controlling the marine plant system according to claim 6, wherein
the marine plant system further comprises:
a storage apparatus configured to store residue generated in the alginate production apparatus; and
a boiler configured to burn the residue, and
when a free space in the storage apparatus is smaller than a predetermined reference value, the controller selects burning of the residue stored in the storage apparatus by the boiler.

10. The method for controlling the marine plant system according to claim 9, comprising supplying calcium carbonate contained in ash generated in the boiler to the intermediate material production apparatus.
